(19) United States
(12) Patent Application Publication
(10) Pub. No.: US 2024/0209310 A1
(43) Pub. Date: Jun. 27, 2024

(54) SYSTEMS AND METHODS FOR THE CONTINUOUS PRODUCTION AND PURIFICATION OF BIOLOGICS

(71) Applicant: UNIVERCELLS TECHNOLOGIES S.A., Nivelles (BE)

(72) Inventors: Jean-Christophe Drugmand, Wavre (BE); Antoine Hubert, Nivelles (BE); Tania Pereira Chilima, Nivelles (BE)

(21) Appl. No.: 18/288,459

(22) PCT Filed: Apr. 29, 2022
(86) PCT No.: PCT/EP2022/061463

§ 371 Date: Oct. 26, 2023

Related U.S. Application Data
(60) Provisional application No. 63/303,133, filed on Jan. 26, 2022.
(60) Provisional application No. 63/181,921, filed on Apr. 29, 2021.

(51) Int. Cl. C12M 1/00; C12M 1/26

(52) U.S. Cl. CPC C12M 47/10; C12M 23/40; C12M 29/04; C12M 29/18; C12M 33/04

(57) ABSTRACT

A system for producing and purifying target biologics is disclosed. A cell culture unit is provided for generating target biologics in a volume of fluid. A purification unit is also provided for purifying the target biologics in the volume of fluid portion directly from the cell culture unit without a clarifying step, such as by using one or more separators, such as expanded beds or chromatography columns arranged in parallel, and potentially operating in series to achieve continuous operation. The one or more separators may include magnetic beads adapted for binding to the target biologics. The purification unit may receive at least a portion of the volume of fluid directly from the cell culture unit, and a collection unit may be provided for collecting the purified target biologics from the purification unit, and also may be adapted for returning the volume of fluid without the target biologics to the cell culture unit. The system may comprise a first vessel adapted to concentrate a volume of fluid including the target biologics, and a second vessel adapted to further concentrating the volume of fluid, the first or second vessels including magnetic beads adapted for binding with the target biologics. Related methods are also disclosed.

This application claims the benefit of U.S. Provisional Patent Application Ser. No. 63/181,921, filed Apr. 29, 2021, and U.S. Provisional Patent Application Ser. No. 63/303,133, filed Jan. 26, 2022, the disclosures of which are incorporated herein by reference. This application further incorporates by reference the following International Patent Applications: WO2019/072584 and WO2020/020569.

### TECHNICAL FIELD

This document relates generally to systems and methods for the continuous production and purification of biologics, such as via linked production, filtration, concentration, and purification processes.

### BACKGROUND

Significant interest exists in the production of biologics for further use in treating medical conditions or research. For instance, a particular current interest relates to biologics known as exosomes, which are extracellular vesicles of a small size (e.g., between 40 and 160 nm). Exosomes are produced by both pathologic and non-pathologic cells, and can be used by cells to exchange substantial information. These vesicles comprise an external phospholipid bilayer, which surrounds a lumen occupied by a variety of proteins and mRNAs. Moreover, proteins are also inserted in the lipid membrane, allowing their affinity purification.

Exosomes and other biologics may be produced using animal cell culturing techniques, and then concentrated/purified by filtration (such as tangential flow filtration (TFF), coated magnetic beads (affinity), packed bed or expanded bed chromatography, or specific purification affinity column or an ultracentrifugation steps). Certain of those processes are not readily scalable, such as ultracentrifugation. Others are insufficient standing alone to guarantee a high concentration factor. Classical process operated those consecutives steps one after the other (batch mode) with intermediate storages, but such processes are long, complex, and costly.

It is possible to accommodate processes including biologic production with in-line, linked tangential flow filtration (TFF) connected to a bioreactor, such as disclosed in International Patent Application Nos. WO2020/020569 and WO2019/072584, the disclosures of which are incorporated herein by reference. However, these arrangements may not be sufficient to reach sufficient targeted concentration and purity level factors desirable for many processes. Thus, end-users would need to rely on additional concentration/purification steps.

Accordingly, a need is identified for improved systems and methods for the production of biologics in an efficient, reliable, and cost-effective manner (such as, for example, using a continuous operation, and optionally without the need for clarification prior to recovery, and also optionally with magnetic or oversized affinity beads for capturing the target biologic). The system would also be customizable and scalable to allow for use in the production of biologics.

### SUMMARY

According to one aspect of the disclosure, a system for producing and purifying target biologics is provided. The system includes a cell culture unit for generating target biologics in a volume of fluid. A purification unit comprises separators, such as expanded beds, or more particularly, a plurality of chromatography columns adapted for expanded bed adsorption, linked to the cell culture unit. Each of the separators is further adapted for receiving at least a portion of the volume of fluid directly from the cell culture unit and purifying the target biologics in the corresponding portion of the volume of fluid, such as in a serial manner (that is, one separator may be online with the cell culture unit for processing/purifying the target biologic, while one or more other separators remain offline, and then one or more separators may be online with the bioreactor for processing/purifying while one or more other separators are offline). A collection unit, such as a harvest vessel, may also be provided for collecting the purified target biologics from the purification unit.

In one embodiment, one or more of the separators include beads with an affinity to the target biologic. The beads may comprise magnetic beads, in which case the system further includes a magnet for attracting the magnetic beads in at least one of the separators (and possibly for each separator present). The beads may comprise oversized or “large” beads, such as having an average diameter of between about 1 μm to about 2000 μm, such as for example about 1 μm to about 1000 μm, or more specifically, about 20 μm to about 500 μm, or any approximate value within these ranges. The oversized beads may have an average radius of between about 0.5 μm to about 1000 μm, or more specifically about 10 μm to about 250 μm.

The cell culture unit may comprise a bioreactor. The bioreactor may be adapted to operate in a perfusion mode or a batch mode. The bioreactor may comprise a fixed bed bioreactor or a stirred tank bioreactor.

In these or another embodiment, at least one of the separators is adapted for returning the portion of the volume of fluid to the cell culture unit following recovery of the target biologics. In a further example, each of the separators is adapted for returning the portion of the volume of fluid to the cell culture unit following recovery of the target biologics.

According to a further aspect of the disclosure, a system for producing and purifying target biologics is provided. The system comprises a cell culture unit for generating target biologics in a volume of fluid. A purification unit is provided for receiving at least a portion of the volume of fluid from the cell culture unit and purifying the target biologics in the volume of fluid portion using magnetic beads adapted to bind with the target biologics. A collection unit is for collecting the purified target biologics from the purification unit.

In one embodiment, the purification unit is adapted for returning the volume of fluid portion to the cell culture unit. The purification unit may comprise one or more chromatography columns, each in fluid communication with the cell culture unit. The purification unit may be adapted for receiving the portion of the volume of fluid directly from the cell culture unit, and may comprise a magnet for attracting the magnetic beads.

Still another aspect of the disclosure is a system for producing and purifying target biologics. The system comprises means for generating target biologics in a volume of fluid, means for serially purifying the target biologics in at least a portion of the volume of fluid received directly from the cell culture unit, and means for collecting the purified target biologics from the purifying means.

In one embodiment, the means for generating comprises a bioreactor. The means for serially purifying may comprise separators, such as a plurality of vessels, for example, chromatography columns connected in parallel to the means for generating target biologics. The means for collecting may comprise a collection unit, such as a vessel.

According to another aspect of the disclosure, a system for producing and purifying target biologics is provided. The system includes a first vessel adapted to concentrate a volume of fluid including the target biologics and a second vessel adapted to further concentrate the volume of fluid. One or both of the first or second vessels include magnetic beads adapted for binding with the target biologics.

In one embodiment, the first vessel comprises a filtration unit adapted for tangential flow filtration. In this or another embodiment, the second vessel comprises a purification unit including the magnetic beads. The first vessel may be adapted to receive the magnetic beads from the first vessel. A magnet may be associated with either or both of the first or second vessels for attracting the magnetic beads.

Either of the first or second vessels may comprise an agitator. A filter downstream of the second vessel may be provided for filtering out the target biologics. A pre-filter may be provided between the first vessel and the second vessel. A bioreactor may be provided for producing target biologics upstream of the first vessel.

A further aspect of the disclosure relates to a system for producing and purifying target biologics. The system comprises means for concentrating a volume of fluid comprising target biologics and means for further concentrating the volume. The means for concentrating the volume may comprise a TFF column or may include magnetic beads, and/or the means for further concentrating the volume comprises a purification unit including magnetic beads. The system may further include means for recycling the magnetic beads from the purification unit to the means for concentrating for repeated use.

Yet another aspect of the disclosure pertains to a method for producing and purifying target biologics. The method comprises concentrating a volume of fluid including target biologics, such as by using tangential flow filtration or magnetic beads. The method also comprises further concentrating the volume of fluid using magnetic beads adapted for binding with the target biologics.

In one embodiment, the step of concentrating the volume is to a concentration factor of at least about 10X. In this or other embodiments, the step of further concentrating the volume is to a concentration factor of up to 5000X. The method may further include the step of re-suspending the magnetic beads following the further concentration step, as well as detaching the target biologics from the magnetic beads.

The method may also involve applying a magnetic field to the magnetic beads prior to or during the step of further concentrating the volume, and filtering the target biologics from the volume following the further concentrating step. The method may also involve the step of filtering the volume to remove unwanted material between the concentrating and further concentrating steps, and using the magnetic beads from the further concentrating step in a different vessel for performing the concentrating step.

Another aspect of the disclosure pertains to a method for recovering target biologics from a volume of fluid in a cell culture unit. The method comprises purifying the target biologics in a portion of the volume of fluid received directly by a plurality of chromatography columns from the cell culture unit. The method further comprises collecting purified target biologics from the portion of the volume of fluid.

The purifying and collecting steps may be performed a plurality of times in parallel. The method may include the step of returning the portion of the volume of fluid to the cell culturing unit without the purified target biologics. At least one of the plurality of chromatography columns comprises magnetic beads.

Another aspect of the disclosure relates to a method for recovering target biologics from a volume of fluid in a cell culture unit. The method comprises purifying the target biologics in a portion of the volume of fluid using magnetic beads adapted for binding with the target biologics, and collecting purified target biologics from the portion of the volume of fluid.

In one embodiment, the purifying and collecting steps are performed a plurality of times in parallel. The method may further include the step of returning the portion of the volume of fluid to the cell culturing unit without the purified target biologics. The purifying step may comprise concentrating the portion of the volume of fluid in a first vessel, which may use tangential flow filtration or magnetic beads, and further concentrating the volume of fluid in a second vessel including the magnetic beads. The concentrating step may comprise using magnetic beads, and the step of recycling the magnetic beads from the second vessel to the first vessel may be performed.

A further aspect of the disclosure relates to a method for recovering target biologics. The method comprises culturing in a bioreactor cells that represent or express the target biologic, and purifying the target biologic by sequentially performing expanded bed adsorption on a different portion of a fluid including the target biologic received directly from the bioreactor without undergoing clarification. The purifying step comprises sequentially delivering the different portions of the fluid independently to each of a plurality of separators or vessels, such as chromatography columns, adapted for performing as expanded beds, and arranged in parallel communication with the bioreactor to allow for substantially continuous operation.

In one example, the purifying step further comprises delivering of the different portions of the fluid to each of the chromatography columns including magnetic beads, restraining the magnetic beads, and recovering the biologic.

A further aspect of the disclosure is a system comprising, in a chained configuration, a cell culture unit for generating extracellular vesicles in a volume of fluid, a concentration unit for concentrating the volume of fluid from the cell culture unit, and a purification unit for purifying the volume of fluid from the concentration unit.

In one embodiment, the purification unit comprises a chromatography column. The purification unit comprises a collector including magnetic beads with an affinity to at least some of the extracellular vesicles.

In another aspect, this disclosure relates to a system for producing exosomes. The system includes a first vessel for concentrating a volume of fluid including exosomes and a second vessel for further concentrating the volume. The second vessel includes magnetic beads adapted for binding with the exosomes.

In one embodiment or example, the first vessel comprises a TFF column. The second vessel comprises a collector including the magnetic beads. The second vessel may comprise a magnet external to the collector for attracting the magnetic beads. The external magnet may be adjacent to a bottom portion of the collector, which may be generally conical.

The collector may comprise an agitator. A filter downstream of the second vessel may be provided for filtering out the exosomes. A pre-filter may be provided between the first vessel and the second vessel. A bioreactor may be provided for producing exosomes upstream of the first vessel, and the first vessel may include an agitator.

Still another aspect of the disclosure relates to a system, comprising means for concentrating a volume of fluid comprising exosomes, and means for further concentrating the volume. In one example, the means for concentrating the volume comprises a TFF column, and the means for further concentrating the volume comprises a collector including magnetic beads.

Yet another aspect of the disclosure is a method for producing exosomes, comprising concentrating a volume of fluid including exosomes, and further concentrating the volume using magnetic beads adapted for binding with the exosomes. The step of concentrating the volume may be to a concentration factor of 10X, and the step of further concentrating the volume may be to a concentration factor of 10X to 100X. The method may further include the step of re-suspending the magnetic beads following the further concentration step, and/or the step of detaching the exosomes from the magnetic beads. Applying a magnetic field to the magnetic beads may be done prior to or during the step of further concentrating the volume. Filtering the exosomes from the volume following the further concentrating step may also be completed, as well as filtering the volume to remove unwanted material between the concentrating and further concentrating steps.

### BRIEF DESCRIPTION OF THE DRAWING FIGURES

FIG. 1 shows a schematic overview of a subsystem with cell culture and concentration units to produce target biologics, such as exosomes.

FIG. 2 shows a schematic overview of one embodiment of a system for producing, purifying and harvesting target biologics including the subsystem of FIG. 1.

FIG. 3 shows a schematic overview of another embodiment of a system for producing, purifying and harvesting target biologics.

FIG. 4 shows a schematic overview of a further embodiment of a system for producing, purifying and harvesting target biologics.

FIG. 5 is a flow chart showing one possible implementation of a method for producing, purifying and harvesting target biologics.

FIG. 6 is a schematic overview of another possible embodiment of a system for producing, purifying and harvesting target biologics.

FIGS. 7, 8, and 9 are schematic views of an exemplary continuous operation of the system of FIG. 6.

### DETAILED DESCRIPTION

In one aspect, this disclosure pertains to a system and method for the production of target biologics at high concentrations. Specifically, the described system and method are designed to allow the purification and the concentration of target biologics produced by an upstream process in bulk. As TFF or magnetic beads as a single step alone would not adequately deal with large volumes generated and concentrate such into small volumes in an efficient or effective manner, the proposal according to one aspect of the disclosure is to provide a system and process that serially performs a several step concentration, potentially in a continuous manner. Consequently, continuous target biologic production at high concentrations may be achieved in a highly efficient manner, as compared to prior approaches, as previously noted.

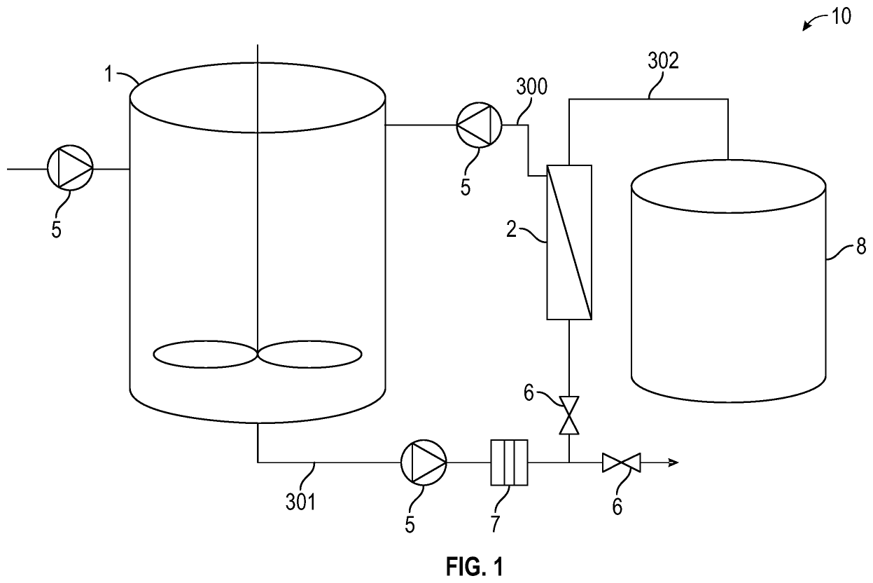

FIG. 1 shows a schematic overview of one example of a general subsystem 10 for producing target biologics, such as for example exosomes in this particular example. This subsystem 10 includes a cell culture unit, such as a bioreactor 1, comprising a cell culture. The bioreactor 1 may be a stirred tank reactor as shown, a fixed bed bioreactor (such as one with a structured fixed bed comprising a non-woven or woven material, or a monolithic fixed bed, such as one made using 3-D printing techniques), or any other type of bioreactor (e.g., a bubble column).

The cell culture may comprise a fluid, such as a volume of liquid media comprising cells for producing biologics of interest, or target biologics. The target biologics may include but are not limited to extracellular vesicles, such as exosomes as noted above. The target biologics may include other biomolecules as well, such as for example nucleic acids (DNA/RNA), viruses (adenovirus, lentivirus, phages, synthetic), viral vectors, virus-like particles, proteins, peptides, eukaryotic cells (human, insect, mammalian, fish, yeast), prokaryotic cells (bacteria gram+, gram−, archebacteria), or other forms of biologics or biomolecules not mentioned or yet to be discovered that may benefit from the disclosed concepts.

In the subsystem, a concentrator 2 is equipped with a retentate line output 300 for collecting the concentrator output and allows re-circulating of the output to an input of the bioreactor 1 in a continuous manner. The bioreactor 1 and concentrator 2 are connected by a conduit 301 facilitating liquid transport from the bioreactor 1 to the concentrator 2. To avoid clogging of the concentrator 2, the liquid may optionally be passed through a pre-filter 7, which may remove solid particles of a certain size from the liquid, but remains permeable to the biologic of interest.

The conduits of the subsystem 10 are fitted with pumps 5 to provide directional liquid flow, for controlling or inducing differential pressure between different parts of the system and to provide crossflow of the liquid through the concentrator 2. In addition, the conduits of the system are provided with valves 6 to control flow distribution, such as for delivery to a downstream collection unit (vessel). The valves 6 further allow for the engagement or disengagement of a specific system segment (e.g., vessel or combination thereof) or conduit.

An output conduit 302 line connects the concentrator 2 with a waste vessel 8 to discard the permeate. This vessel 8 may comprises at least one waste container (such as a tank) where undesired material produced in the system or by-products of the process can be temporarily stored. The waste vessel 8 may also function as a decontamination vessel, and may include for instance a heater, as described in International Patent Application WO2020079274, the disclosure of which is incorporated herein by reference.

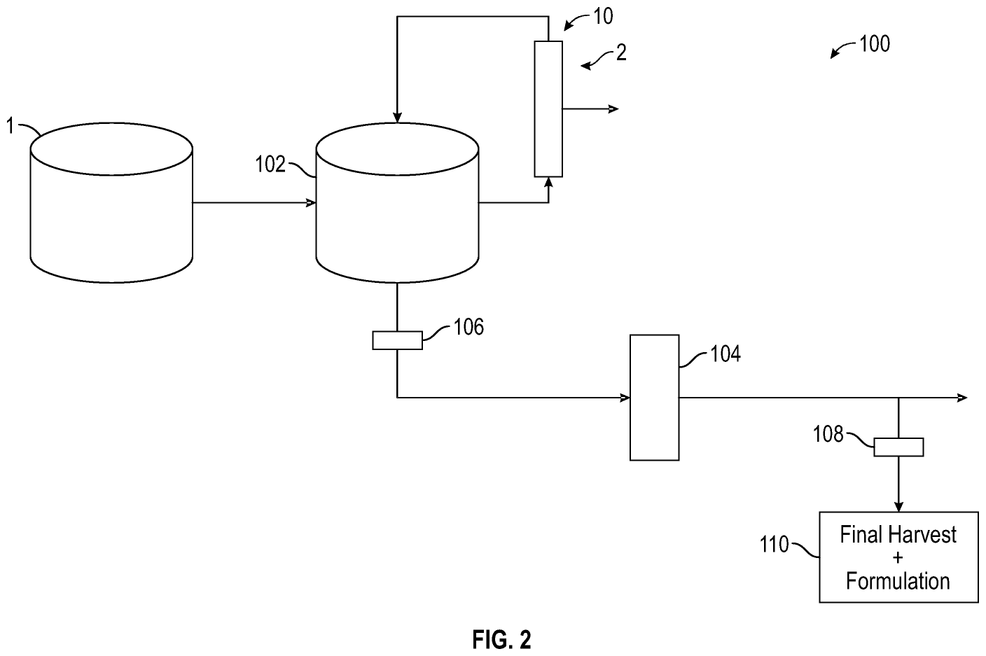

Turning to FIG. 2, this subsystem 10 may form part of a larger system 100 for harvesting the produced target biologics in an efficient, and potentially continuous manner. For example, the system 100 includes a harvest vessel 102 as the concentrator 2. This vessel 102 may be used to receive a bulk product or bulk comprised of a volume of fluid including target biologics and achieve a volume reduction of a factor (e.g., 10X), such as using TFF (e.g., in-line TFF or a bioharvest concentration vessel).

Thereafter, beads coated with specific antibodies or any dedicated ligand chemistry (e.g., directed against surface antigens) may be added to the bulk to form a solution, or slurry, with the beads in suspension. These so-called “affinity” beads are designed to bind the biologics of interest. To ensure that the desired binding is achieved, the slurry may also be agitated or mixed. This may be achieved, for example, by associating an agitator with the harvest vessel 102.

The volume of fluid including the beads is then transferred downstream to a purification unit 104, which may comprise a vessel. An optional pre-filter 106 may be used to remove cellular debris prior to transfer to the purification unit 104.

The beads may be magnetic (that is, susceptible to attraction by an external magnetic field) and, thus, in the purification unit 104, a magnetic field may be used to attract the magnetic beads and allow the removal of the excess volume by further decreasing it (e.g., by 10-100X concentration factor, but potentially greater, such as from generally about 10X up to any number in the range of 1000X-5000X). This magnetic field may be provided by a magnet M, which may be located on or in a wall of the purification unit 104. In the provided illustration, the magnet M is arranged adjacent and external to the bottom portion of the purification unit 104, which may be generally conical as shown. Optional agitation may also be provided by associating an agitator with the purification unit 104.

The magnetic beads may then be re-suspended. This may be done, for instance, using a detachment solution, such as a low pH buffer or other manner of causing an affinity change (e.g., conductivity) supplied to the purification unit 104 to detach smoothly the target biologics from the beads. The magnetic beads are then separated from the target biologics using the magnet M. The pH may then be restored to a higher (physiological) level (e.g., 7.0-7.4), such as by the addition of a suitable (e.g. acidic) buffer. A final filtration through a filter 108 (e.g., 0.22 μm pore size, but other pore sizes may be useful) ensures that no magnetic beads accidentally remain in the final volume comprising the harvested target biologics, which may be delivered to a collection unit (vessel) 110.

Summarizing the foregoing, the proposed system 100 and method described above combine concentration and purification tools to achieve the production and purification of target biologics in an efficient manner. Specifically, a first vessel, such as a harvest vessel 102 comprising a TFF column, is used to decrease the volume of fluid by a first (e.g., around 10X) factor. This is followed directly by a purification with affinity magnetic beads in a purification unit 104, which will allow a volume reduction of second, higher (e.g., greater than around 10X) factor. These steps may be followed by further processing (e.g., separation and filtration) to obtain the target biologics. All the processes occur in a succession of serial events, and may be done in a continuous fashion to increase target biologic production.

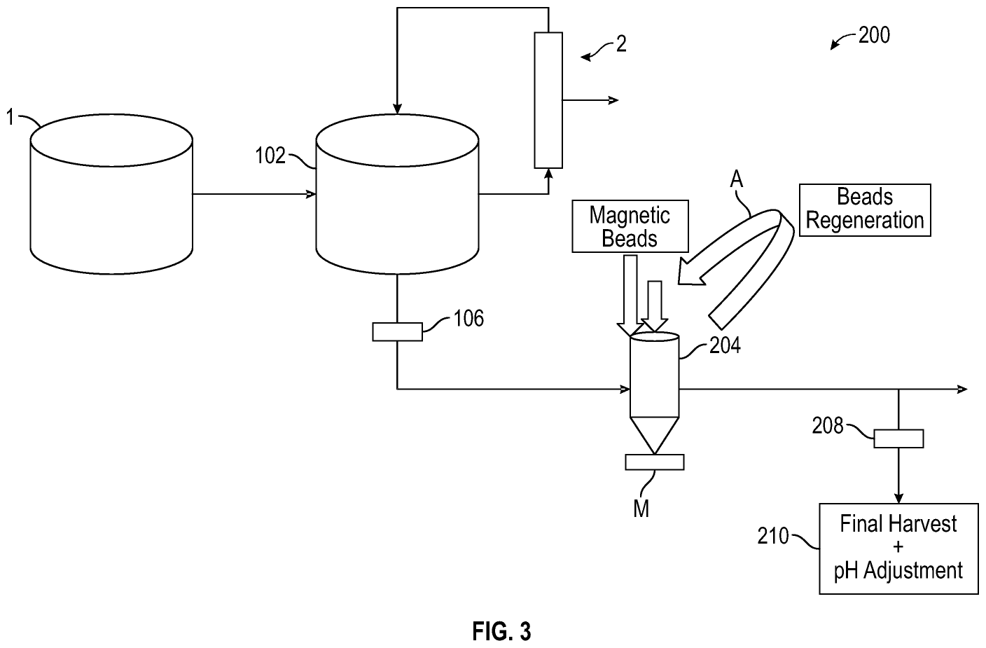

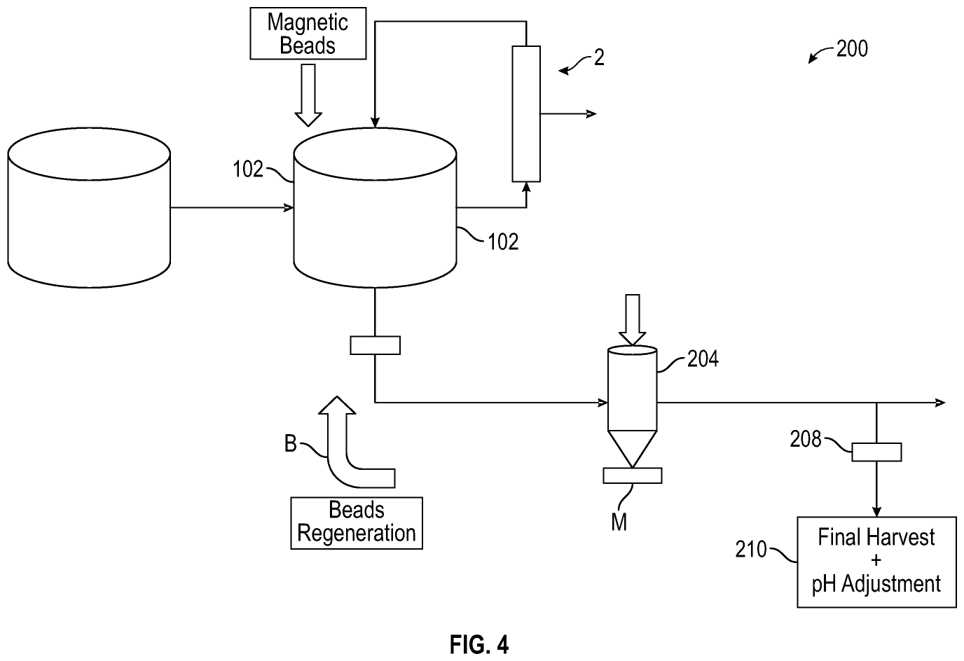

Turning to FIG. 3, a further embodiment of a system 200 is shown that may use a purification unit, such as one involving the use of magnetic beads coated with specific antibodies or ligands (e.g., ligands directed toward surface antigens). These magnetic beads may be added to the target biologic following production in the bioreactor 1, such as for example in the fluid of the harvest vessel 102 (see FIG. 4), to form a solution, or slurry, with the beads in suspension. Alternatively, the magnetic beads may be introduced into a downstream purification unit 204, such as a chromatography column, as outlined further in the following description. These so-called affinity magnetic beads may comprise, for example, iron atoms surrounded or covered by ligands designed to bind to the biologic of interest, and thus may allow for enhanced selectivity.

Once the magnetic beads and target biologics are present in the purification unit 204, a magnetic field is initiated proximate to the purification unit 204 to attract the magnetic beads and retain them in the collector while the excess volume is further decreased (e.g., by 10-100X concentration factor). This magnetic field may be provided by a magnet M arranged adjacent and external to the bottom portion of the purification units 204, which may be generally conical as shown (which may aid in collecting the magnetic beads). The magnet M may be, for example, a permanent magnet movable toward and away from the purification unit 204 to generate the desired magnetic field for restraining the magnetic beads, or alternatively a non-permanent magnet (e.g., an electromagnet that can be activated selectively for restraining the beads and deactivated when such is not desired). The magnet M may also be integrated into the purification unit 204. As noted below, optional agitation may also be provided by associating an agitator (e.g., a stirred rod, impeller or shaker mechanism) with the purification unit 204.

Waste, such as supernatant without the target biologics, may then be removed from the purification unit 204, such as by using a pump. The magnetic beads may then be re-suspended in the purification unit 204. This may be done, for instance, by releasing the applied magnetic field and using a detachment solution, such as a low pH buffer or other manner of causing an affinity change (e.g., conductivity) supplied to the purification unit 204 to detach the target biologics from the beads without impacting the stability of the target biologics.

The magnetic beads may then be retained using the applied magnetic field, with the target biologics released in a subsequent fluid flush or drain. The pH may then be restored to a higher (physiological) level (e.g., 7.0-7.4), such as by the addition of an appropriate buffer to the purification unit 204. The target biologic-containing fluid may then be exhausted to a final filtration step, such as by using suitable valves to pass fluid from the purification unit 204 through a filter 208 (e.g., 0.22 μm pore size, but other pore sizes may be useful). This filter 208 ensures that no magnetic beads accidentally remain in the final volume comprising the harvested target biologics, which again may be delivered to a collection unit 210.

The magnetic beads associated with purification unit 204 may be used for multiple purification cycles, or regenerated, as indicated by action arrow A in FIG. 3. Alternatively, or additionally, the beads may be recycled by returning them to the upstream harvest vessel 102, as shown in FIG. 4. This is indicated by action arrow B. This recycling may be achieved in an automated manner using a bead return conduit or loop as part of the chained system 200 or associated process. However, it is also possible to recover the magnetic beads from the purification unit 204 and manually reintroduce them to the harvest vessel 102.

To ensure that the desired binding is achieved, the slurry may also be agitated or mixed in the harvest vessel 102, such as by using an associated agitator (which may comprise, for example, a non-contact drive, such as a magnetically driven stir bar, impeller or shaker mechanism, which may provide gentle agitation so as to avoid creating undesirable shear stresses). The mixed fluid including the magnetic beads is then transferred downstream to the purification unit 204. Additionally or alternatively, agitation may also be applied to the purification unit 204.

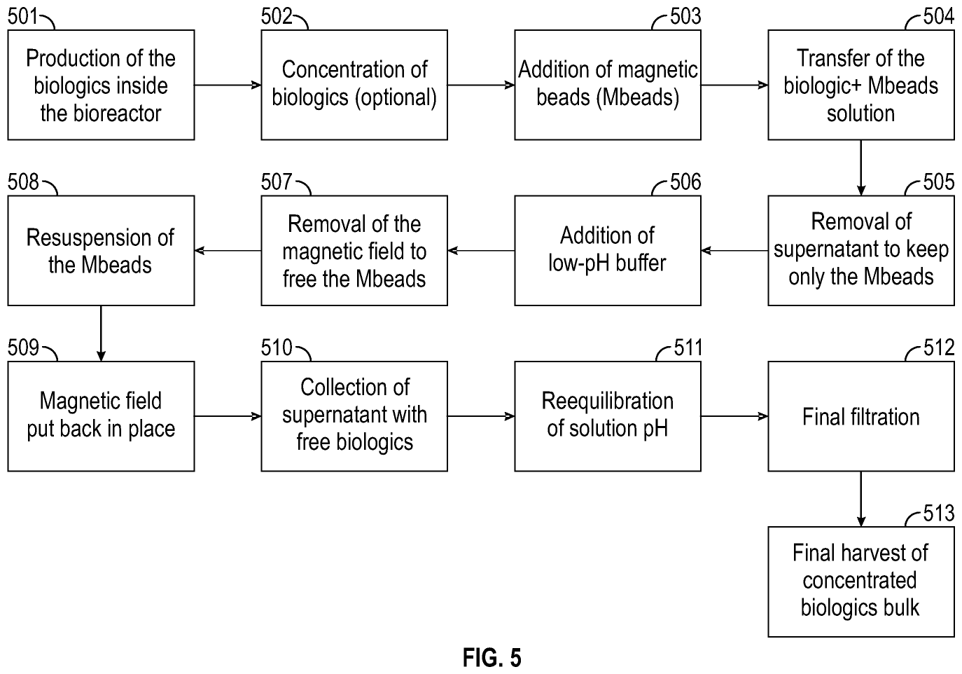

FIG. 5 is a flow chart illustrating one exemplary process for harvesting and purifying target biologics. The process may involve at step 501 the production of the target biologics in the bioreactor (for example, as they are secreted and the media perfused, they may be continuously sent to a harvest vessel). At step 502, an optional concentration of target biologics is implemented, such as via TFF in a harvest bottle. At step 503, magnetic beads are added when the final volume is reached, possibly in parallel with gentle mixing.

At step 504, the solution including the target biologics and magnetic beads is transferred to a purification unit with a magnet (e.g., adjacent to the bottom). In step 505, the supernatant is removed resulting in only the magnetic beads in the collector. Step 506 involves the addition of a detachment solution, such as a low pH (e.g., 5.0) buffer or other manner of causing an affinity change (e.g., conductivity), to the beads to detach the biologics of interest from their antibodies (linked to the beads covalently).

At step 507, the magnetic field is removed to free the magnetic beads inside of the collector. Step 508 involves the resuspension of the beads within the added buffer (e.g., low pH). At step 509, the magnetic field is reintroduced, and step 510 involves the collection of supernatant with freed target biologics. Step 511 involves the re-equilibration of the solution (such as to reach a physiological value in the case of a low pH buffer), followed by step 512, final filtration with filter (e.g., 0.22 μm pores) for purification, and at step 513, final harvest of concentrated bulk of target biologics.

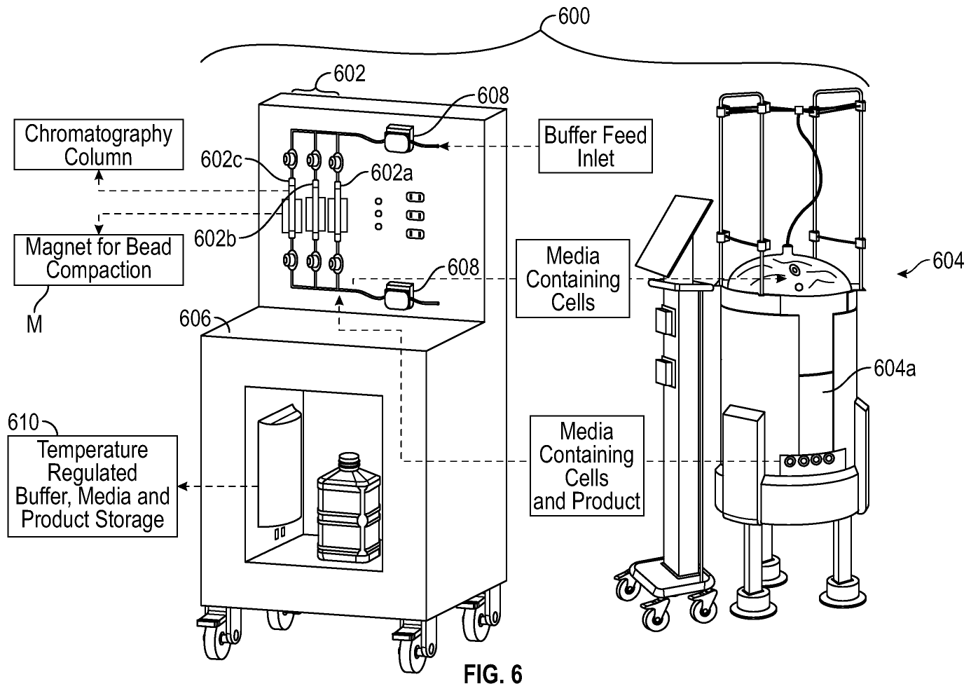

Turning now to FIG. 6, a further embodiment of a system 600 that may employ a purification unit 602 for performing expanded bed adsorption to recover a target biologic in a continuous manner from a fluid including the target biologic without undergoing clarification. The system 600 may comprise one or more separators, such as vessels in the form of chromatography columns (which is meant to be broadly interpreted to cover traditional columnar structures, as well as other shapes of vessels (e.g., cubic) that are not necessarily columnar in nature). In one example, three such vessels in the form columns 602a, 602b, 602c are provided, but the number could be fewer or more than three (one, four, or more—the number and size depends on the particular process conditions, with the goal for the total volume of the purification unit to correspond to that of the cell culture unit), each in direct communication with a cell culture unit. Specifically, the columns 602a, 602b, 602c are arranged for independent parallel communication with an upstream bioreactor 604, which may comprise one having a fixed bed 604a therein, or alternatively may be a stirred tank version (see FIG. 1). The columns 602a, 602b, 602c may form part of a station or skid 606, which may also include a controller for controlling the operation of the system 600. Each of the columns 602a, 602b, 602c may be associated with magnets M for use in connection with magnetic beads located therein, as previously noted.

Suitable pumps 608 and conduits for transmitting fluid, as well as a source 610 of temperature-regulated buffer may also be provided, which buffer may communicate via a pump 608 with the top of each column 602a, 602b, 602c. The bottom of the columns 602a, 602b, 602c may be in two-way communication with the bioreactor 604 via additional pump(s) 608 for receiving media containing cells and product (e.g., the target biologics), and optionally returning the cells once the product is recovered from the columns, as outlined further in the following description (but the cells could also be sent to a waste container or vessel). Control of the operation of the pumps 608 and valves may be provided by a controller, which may be part of the station or skid 606 or a separate device for providing instructions. Suitable sensors may also be used to detect volumes and control the flow accordingly.

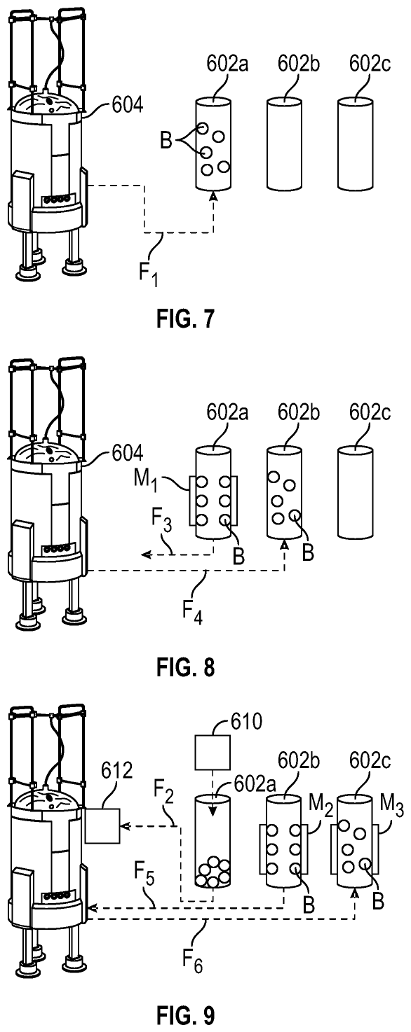

FIGS. 7, 8 and 9 illustrate an exemplary operation of the system is described. As shown in FIG. 7, media feed F1 from the bioreactor 604 containing product and cells reflecting a portion of the volume of fluid therein is delivered to a first column 602a. The bioreactor 604 operation may be in either batch mode or perfusion mode, with an optional surge tank, as well as filters upstream or downstream of the purification unit 602, as described above. This media feed may be mixed into the column 602a, such as by delivery from the bottom of the column (but it could be another location, such as the top). If the bottom, this pushes magnetic beads B upwards in order to create mixing, thereby enabling the product (target biologic) to contact the ligands in the beads. An outlet of the column 602a at the top may remain closed. While a particular example is shown and described, it can be understood that any expanded bed adsorption (EBA) technique may be used to achieve separation.

As shown in FIG. 8, magnetic field may then be applied to the first column 602a via an associated magnet M1. The applied magnetic field serves to restrain the beads B with the product attached to them, such as by causing them to stick to the adjacent walls of the column 602a. The product may then be recovered from the first column 602a and a corresponding feed F2 delivered to a collection unit 612. Once completed, media feed F3 containing the cells flowed back to the bioreactor. Buffer from a source 610 is used to wash and elute the product in the first column 602a, such as in a top to bottom direction to take advantage of gravitational forces (but again could be from the bottom or another location) to direct the beads B to the bottom of the column (alternatively, radial flow can be used to elute and wash the beads or the magnetic field can be used to sediment the beads at the bottom of the column).

Using suitable valves, media feed F4 from the bioreactor 604 is now redirected to the second column 602b, while the first column 602a is regenerated. A magnetic field is applied to the second column 602b via an associated magnet M2, restraining the beads B therein with the product attached, such as by causing them stick to the walls of this column. Product may then be recovered and sent to the collection unit 612 in like manner, and media feed F3 containing the cells may be returned to the bioreactor 604.

Media feed F4 with cells and product from the bioreactor 604 may be redirected to the third column 602c, while the second column 602b is regenerated. The above process for separating the target biologic using the magnetic beads B may be repeated using the third column 602c, including by using an associated magnet M3 to attract the beads B therein. Product recovered may be delivered to the collection unit 612, and cells and media returned to the bioreactor 604.

In this manner, a continuous process of product recovery and return of the cells to the bioreactor 604 may be achieved. In view of the parallel processing made possible by the concurrent use of plural columns to separate the target biologic, the need for concentration as an intermediate step in the process may be avoided. Any requirement for clarification is also avoided by the use of magnetic beads.

While affinity chromatography using expanded bed adsorption is mentioned above, different approaches could be taken. For example, ion exchange chromatography, size exclusion chromatography, hydrophobic interaction chromatography, hydroxyapatite/fluorapatite chromatography, or other known suitable forms for separating biologics of interest from a volume of fluid including cells.

Likewise, magnetic beads are mentioned, but so-called oversized or “large” beads may be used in connection with a purification unit. This refers to beads having average diameters of between about 1 μm to about 2000 μm, or more specifically, about 20 μm to about 1000 μm. In one embodiment, at least about 80% of the beads have a diameter of about 200 μm to about 500 μm, or at least about 85%, or at least about 90% of the beads have a diameter of about 200 μm to about 500 μm. Such beads may have an average radius of between about 0.5 μm to about 1000 μm, or about 100 μm to about 250 μm. Examples of such alternative beads are described in U.S. Patent Application Publication No. 2019/0176127 and U.S. Pat. No. 5,466,377, the disclosures of which are incorporated herein by reference.

When such oversized beads are packed into a column, interstitial channels are formed. These channels are wide enough to allow the cells and cell fragments to pass through the bed without clogging and when the channels are free of smaller beads which, due to their size, could restrict or block the passage of cells and cell fragment, the user can utilize the packed beads to avoid deleterious filtration or centrifugation, precipitation or other costly, time-consuming and potentially product-losing steps prior to the chromatography purification step(s).

With oversized beads, the surface area per volume is lower so that the efficiency of capture of the target biomolecule may be less. However, this is traded for the benefit of greater interstitial spacing and its advantages to avoid clogging and the need for a costly clarification step. The magnetic solution can avoid the tradeoff by providing smaller beads with greater surface area while also allowing for the avoidance of clogging and clarification step due to magnetic attraction/spacing.

Summarizing this disclosure, it may relate to any one of the following items in any ordered combination even if not expressly noted:
1. A system for producing and purifying target biologics, comprising:
   a cell culture unit for generating target biologics in a volume of fluid;
   a purification unit comprising separators, such as a plurality of purification or separation vessels, for example, expanded beds or chromatography columns, each adapted for expanded bed adsorption, and linked to the cell culture unit, each of the separators adapted for receiving at least a portion of the volume of fluid directly from the cell culture unit, such as in a serial manner, and purifying the target biologics in the corresponding portion of the volume of fluid; and
   a collection unit for collecting the purified target biologics from the purification unit.
2. The system of item 1, wherein the separators, such as expanded beds or chromatography columns, include beads with an affinity to the target biologic.
3. The system of item 2, wherein the beads comprise magnetic beads, and further including a magnet for attracting the magnetic beads.
4. The system of item 2 or item 3, wherein the beads comprise oversized beads.
5 The system of item 4, wherein the oversized beads have an average diameter of between about 1 μm to about 1000 μm.
6. The system of any of items 1-5, wherein the cell culture unit comprises a bioreactor adapted to operate in a perfusion mode or a batch mode.
7. The system of item 6, wherein the bioreactor comprises a fixed bed bioreactor or a stirred tank bioreactor.
8. The system of any of items 1-7, wherein at least one of the chromatography columns is adapted for returning the portion of the volume of fluid to the cell culture unit following recovery of the target biologics.
9 The system of any of items 1-7, wherein each of the chromatography columns is adapted for returning the portion of the volume of fluid to the cell culture unit following recovery of the target biologics.
10. A system for producing and purifying target biologics, comprising:
   a cell culture unit for generating target biologics in a volume of fluid;
   a purification unit for receiving at least a portion of the volume of fluid from the cell culture unit and purifying the target biologics in the volume of fluid portion using magnetic beads adapted to bind with the target biologics; and
   a collection unit for collecting the purified target biologics from the purification unit.
11. The system of item 10, wherein the purification unit is adapted for returning the volume of fluid portion to the cell culture unit.
12. The system of item 10 or item 11, wherein the purification unit comprises one or more chromatography columns, each in fluid communication with the cell culture unit.
13. The system of item 12, wherein the purification unit is adapted for receiving the portion of the volume of fluid directly from the cell culture unit.
14. The system of item 12, wherein the purification unit comprises a magnet for attracting the magnetic beads.
15. A system for producing and purifying target biologics, comprising:
   means for generating target biologics in a volume of fluid;
   means for serially purifying the target biologics in at least a portion of the volume of fluid received directly from the cell culture unit; and
   means for collecting the purified target biologics from the purifying means.
16. The system of item 15, wherein the means for generating comprises a bioreactor.
17. The system of item 15 or item 16, wherein the means for serially purifying comprises a plurality of chromatography columns connected in parallel to the means for generating target biologics.
18. The system of any of items 15-17, wherein the means for collecting comprises a collection unit.
19. A system for producing and purifying target biologics, comprising:
   a first vessel adapted to concentrate a volume of fluid including the target biologics; and
   a second vessel adapted to further concentrate the volume of fluid, the first vessel or second vessel including magnetic beads adapted for binding with the target biologics.
20. The system of item 19, wherein the first vessel comprises a filtration unit including a tangential flow filter.
21. The system of item 19 or item 20, wherein the second vessel comprises a purification unit including the magnetic beads.
22. The system of item 19, wherein the first vessel is adapted to receive the magnetic beads from the first vessel.
23. The system of any of items 19-22, further including a magnet for attracting the magnetic beads.
24. The system of any of items 19-23, wherein either of the first or second vessels comprises an agitator.
25. The system of any of items 19-24, further including a filter downstream of the second vessel for filtering out the target biologics.
26. The system of any of items 19-25, further including a pre-filter between the first vessel and the second vessel.
27. The system of any of items 19-25, further including a bioreactor for producing target biologics upstream of the first vessel.
28. A system for producing and purifying target biologics, comprising:
   means for concentrating a volume of fluid comprising target biologics; and
   means for further concentrating the volume.
29. The system of item 28, wherein:
   the means for concentrating the volume comprises a TFF column; and
   the means for further concentrating the volume comprises a purification unit including magnetic beads.
30. The system of item 28 or item 29, further including means for recycling the magnetic beads from the purification unit to the means for concentrating.
31. A method for producing and purifying target biologics, comprising:
   concentrating a volume of fluid including target biologics; and
   further concentrating the volume of fluid using magnetic beads adapted for binding with the target biologics.
32. The method of item 31, wherein the step of concentrating the volume is to a concentration factor of at least about 10X.
33. The method of item 31 or item 32, wherein the step of further concentrating the volume is to a concentration factor of up to 5000X.
34. The method of any of items 31-33, further including the step of re-suspending the magnetic beads following the further concentration step.
35. The method of any of items 31-34, further including the step of detaching the target biologics from the magnetic beads.
36. The method of any of items 31-35, further including the step of applying a magnetic field to the magnetic beads prior to or during the step of further concentrating the volume.
37. The method of any of items 31-36, further including the step of filtering the target biologics from the volume following the further concentrating step.
38. The method of any of items 31-37, further including the step of filtering the volume to remove unwanted material between the concentrating and further concentrating steps.
39. The method of any of items 31-38, further including the step of using the magnetic beads from the further concentrating step in a different vessel for performing the concentrating step.
40. A method for recovering target biologics from a volume of fluid in a cell culture unit, comprising:
   purifying the target biologics in a portion of the volume of fluid received directly by a plurality of chromatography columns from the cell culture unit; and
   collecting purified target biologics from the portion of the volume of fluid.
41. The method of item 40, wherein the purifying and collecting steps are performed a plurality of times in parallel.
42. The method of any of items 40-41, further including the step of returning the portion of the volume of fluid to the cell culturing unit without the purified target biologics.
43. The method of any of items 40-42, wherein at least one of the plurality of chromatography columns comprises magnetic beads.
44. A method for recovering target biologics from a volume of fluid in a cell culture unit, comprising:
   purifying the target biologics in a portion of the volume of fluid using magnetic beads adapted for binding with the target biologics; and
   collecting purified target biologics from the portion of the volume of fluid.
45. The method of item 4, wherein the purifying and collecting steps are performed a plurality of times in parallel.
46. The method of any of items 44-45, further including the step of returning the portion of the volume of fluid to the cell culturing unit without the purified target biologics.
47. The method of any of items 44-46, wherein the purifying step comprises:
   concentrating the portion of the volume of fluid in a first vessel; and further concentrating the volume of fluid in a second vessel including the magnetic beads.
48. The method of item 47, wherein the concentrating step comprising using magnetic beads.
49. The method of item 47 or item 48, further including the step of recycling the magnetic beads from the second vessel to the first vessel.
50. A method for recovering target biologics, comprising:
   culturing in a bioreactor cells that represent or express the target biologic; and
   purifying the target biologic by sequentially performing expanded bed adsorption on a different portion of a fluid including the target biologic received directly from the bioreactor without undergoing clarification.
51. The method of item 50, wherein the purifying step comprises sequentially delivering the different portions of the fluid independently to each of a plurality of chromatography columns arranged in parallel communication with the bioreactor to allow for substantially continuous operation.
52. The method of item 51, wherein the purifying step further comprises:
   delivering of the different portions of the fluid to each of the chromatography columns including magnetic beads;
   restraining the magnetic beads; and
   recovering the biologic.
53. A system comprising, in a chained configuration:
   a cell culture unit for generating extracellular vesicles in a volume of fluid;
   a concentration unit for concentrating the volume of fluid from the cell culture unit; and
   a purification unit for purifying the volume of fluid from the concentration unit.
54. The system of item 53, wherein the purification unit comprises a chromatography column.
55. The system of item 53, wherein the purification unit comprises a collector including magnetic beads with an affinity to at least some of the extracellular vesicles.
56. A system for producing exosomes, comprising:
   a first vessel for concentrating a volume of fluid including exosomes; and
   a second vessel for further concentrating the volume, the second vessel including magnetic beads adapted for binding with the exosomes.
57. The system of item 56, wherein the first vessel comprises a TFF column.
58. The system of item 56 or item 57, wherein the second vessel comprises a collector including the magnetic beads.
59. The system of item 58, wherein the second vessel comprises a magnet external to the collector for attracting the magnetic beads.
60. The system of item 59, wherein the external magnet is adjacent to a bottom portion of the collector.
61. The system of any of items 58-60, wherein the bottom portion of the collector is generally conical.
62. The system of any of items 58-61, wherein the collector comprises an agitator.
63. The system of any of items 56-62, further including a filter downstream of the second vessel for filtering out the exosomes.
64. The system of any of items 56-63, further including a pre-filter between the first vessel and the second vessel.
65. The system of any of items 56-64, further including a bioreactor for producing exosomes upstream of the first vessel.
66. The system of any of items 56-65, wherein the first vessel includes an agitator.
67. A system, comprising:
   means for concentrating a volume of fluid comprising exosomes; and
   means for further concentrating the volume.
68. The system of item 67, wherein:
   the means for concentrating the volume comprises a TFF column; and
   the means for further concentrating the volume comprises a collector including magnetic beads.
69. A method for producing exosomes, comprising:
   concentrating a volume of fluid including exosomes; and further concentrating the volume using magnetic beads adapted for binding with the exosomes.
70. The method of item 69, wherein the step of concentrating the volume is to a concentration factor of 10X.
71. The method of item 69 or item 70, wherein the step of further concentrating the volume is to a concentration factor of 10X to 100X.
72. The method of any of items 69-71, further including the step of re-suspending the magnetic beads following the further concentration step.
73. The method of any of items 69-72, further including the step of detaching the exosomes from the magnetic beads.
74. The method of any of items 69-73, further including the step of applying a magnetic field to the magnetic beads prior to or during the step of further concentrating the volume.
75. The method of any of items 69-74, further including the step of filtering the exosomes from the volume following the further concentrating step.
76. The method of any of items 69-75, further including the step of filtering the volume to remove unwanted material between the concentrating and further concentrating steps.

As used herein, the following terms have the following meanings:
“A”, “an”, and “the” as used herein refers to both singular and plural referents unless the context clearly dictates otherwise. By way of example, “a compartment” refers to one or more than one compartment.
“About,” “substantially,” “generally” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.
“Comprise”, “comprising”, “comprises” and “comprised of” as used herein are synonymous with “include”, “including”, “includes” or “contain”, “containing”, “contains” and are inclusive or open-ended terms that specifies the presence of what follows, e.g., “component includes” does not exclude or preclude the presence of additional, non-recited components, features, element, members, steps, known in the art or disclosed therein.

While preferred embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the protection under the applicable law and that methods and structures within the scope of these claims and their equivalents be covered thereby.

1. A system for producing and purifying target biologics, comprising:
a cell culture unit for generating target biologics in a volume of fluid;
a purification unit comprising a plurality of separators linked to the cell culture unit, each of the separators adapted for receiving at least a portion of the volume of fluid directly from the cell culture unit and purifying the target biologics in the corresponding portion of the volume of fluid; and
a collection unit for collecting the purified target biologics from the purification unit.

2. The system of claim 1, wherein the separators comprise expanded beds.

3. The system of claim 2, wherein the separators comprise chromatography columns, each including beads with an affinity to the target biologic.

4. The system of claim 3, wherein the beads comprise magnetic beads, and further including a magnet for attracting the magnetic beads.

5. The system of claim 3, wherein the beads comprise oversized beads having an average diameter of between about 1 μm to about 1000 μm.

6. The system of any of claims 1-5 claim 1, wherein the cell culture unit comprises a bioreactor adapted to operate in a perfusion mode or a batch mode.

7. The system of claim 6, wherein the bioreactor comprises a fixed bed bioreactor or a stirred tank bioreactor.

8. The system of claim 1, wherein at least one of the separators is adapted for returning the portion of the volume of fluid to the cell culture unit following recovery of the target biologics.

9. The system of claim 1, wherein each of the separators is adapted for returning the portion of the volume of fluid to the cell culture unit following recovery of the target biologics.

10. A system for producing and purifying target biologics, comprising:
a cell culture unit for generating target biologics in a volume of fluid;
a purification unit for receiving at least a portion of the volume of fluid from the cell culture unit and purifying the target biologics in the volume of fluid portion using magnetic beads adapted to bind with the target biologics; and
a collection unit for collecting the purified target biologics from the purification unit.

11. The system of claim 10, wherein the purification unit is adapted for returning the volume of fluid portion to the cell culture unit.

12. The system of claim 10, wherein the purification unit comprises one or more separators, such as expanded beds or chromatography columns, each in fluid communication with the cell culture unit.

13. The system of claim 12, wherein the purification unit is adapted for receiving the portion of the volume of fluid directly from the cell culture unit.

14. The system of claim 12, wherein the purification unit comprises a magnet for attracting the magnetic beads.

15. A system for producing and purifying target biologics, comprising:
means for generating target biologics in a volume of fluid;
means for serially purifying the target biologics in at least a portion of the volume of fluid received directly from the cell culture unit; and
means for collecting the purified target biologics from the purifying means.

16. The system of claim 15, wherein the means for generating comprises a bioreactor.

17. The system of claim 15, wherein the means for serially purifying comprises a plurality of separators, such as expanded beds or chromatography columns, connected in parallel to the means for generating target biologics.

18. The system of claim 15, wherein the means for collecting comprises a collection unit, such as a vessel.

19.-76. (canceled)
